(19) United States
(12) Patent Application Publication
(10) Pub. No.: US 2003/0229027 A1
(43) Pub. Date: Dec. 11, 2003

(54) Stabilized natural cannabinoid formulation

(76) Inventors: Anko C. Eissens, Weesp (NL); Dirk Jan Van Drooge, Weesp (NL); Woulter L.J. Hinrichs, Weesp (NL); Henderik W. Frijlink, Weesp (NL)

Correspondence Address: MAYER, BROWN, ROWE & MAW, P.O. BOX 2828, CHICAGO, IL, 60690, US

(21) Appl. No.: 10/405,320

(22) Filed: Apr. 2, 2003

Related U.S. Application Data
(60) Provisional application No. 60/369,613, filed on Apr. 3, 2002.

(51) Int. Cl. A61K 31/70; A61K 31/353; A61K 31/16

(52) U.S. Cl. 514/23; 514/454; 514/455; 514/627

(57) ABSTRACT

The present invention is related to a pharmaceutical composition comprising a natural cannabinoid compound, for example, Δ⁹-tetrahydrocannabinol, and a glass of a sugar, sugar alcohol, mixture of sugars or mixture of sugar alcohols, characterized in that the natural cannabinoid compound is incorporated in the sugar glass as a monomolecular encapsulation without formation of a guest-host complex. The invention further relates to a method for the preparation of the pharmaceutical composition in the form of a sugar glass by freeze drying, spray drying, vacuum drying, or critical drying of a stable mixture containing the natural cannabinoid compound and a sugar or a mixture of sugars.

### RELATED CASE INFORMATION

This application claims priority to U.S. Provisional Application Serial No. 60/369,613, filed 3 Apr. 2002, which is incorporated herein by reference to the extent permitted by law.

### BACKGROUND

The present invention is related to a pharmaceutical formulation stabilizing natural cannabinoid compounds, especially Δ⁹-tetrahydrocannabinol (THC). The invention further relates to a method for preparing the formulations.

Natural cannabinoid compounds, which can be obtained from several natural sources, but that are normally obtained from Cannabis Sativa, can be used as a therapeutic agent for the treatment of a large variety of diseases. For an overview of natural cannabinoid compounds see David T. Brown ed., Cannabis, Harwood Academic Publishers 1998, ISBN 90-5702-291-5. An example of a natural cannabinoid compound is THC, which is on the market as Marinol® (generic name dronabinol). Currently, THC is formulated as a soft gelatin capsule for oral administration in which the drug is dissolved in an oil. The disadvantage is that in this formulation THC is not stable. As a consequence, it has to be stored at low temperatures (4° C.). It is clear that a low stability of a compound and the need to store the pharmaceutical formulation in the refrigerator is a serious drawback for a pharmaceutical product.

WO9932107 discloses the use of cyclodextrins for solubilization of THC in a biphasic delivery system or a microsphere delivery system. The solubilizing action of cyclodextrins is caused by the formation of so-called inclusion complexes or guest-host complexes. The object of the subject matter of WO9932107 is the solubilization of THC in order to promote absorption from the nasal cavity. Nothing is disclosed in the application about stability of the formulated THC. Thus, nothing can be concluded about the stabilizing effect of the formation of the guest-host complex as it is known to the person skilled in the art that these complexes sometimes have a stabilizing effect but in other cases lead to deterioration of the active compound due to catalytic effects. Further cyclodextrins have the draw-back of causing mucosal irritation when applied as a nasal or pulmonary formulation. Especially cyclodextrin derivatives which have surfactant properties are irritant for mucosal tissues.

WO9736577 describes the use of dry solid lipid compositions useful for the oral delivery of lipohilic compounds such as natural cannabinoids, the solid lipid composition comprising, apart from the active substance, a solid fat and a phospholipid. The aim of this composition is the enhancement of oral bioavailability and not the enhancement of the stability of the active substance.

WO0078817 discloses the stabilization of alkaline phosphatase by drying the protein from a pure aqueous solution in the presence of inulin, an oligosaccharide. During drying, the protein is encapsulated monomolecularly by a matrix consisting of amorphous inulin, which is in a glassy state. Amongst other things stabilization is achieved because the protein is vitrified and is shielded from its environment. Alkaline phosphatase is, however, a hydrophilic compound which is very soluble in water and it can be formulated directly from an aqueous solution. Further the stabilization relates especially to the preservation of the tertairy and quaternary structure of the protein, which is important for the enzymatic activity.

WO 9118091 describes the use of non-reducing sugar molecules, especially monoglycosides like maltitol, lactitol and palatinit for the preservation of stability of enzymes, such as restriction endonuclease Pst I, and antibodies, which are hydrophilic compounds. According to this patent application stabilized enzymes can be prepared by mixing of the enzyme with the sugar and a proprietary buffer, followed by air drying. This method cannot be used for lipophilic compounds, as these compounds cannot be solved in sufficient amounts in a polar system. Maltitol and lactitol have glass transition temperatures of 44° C. (Y. Roos, Carbohydrate Research 1993, 238, 39-48) resp. 33° C. at dry conditions.

Therefore, there is a need for stable formulations of unstable natural cannabinoid compounds like THC, which can be stored, for example, at ambient conditions for prolonged times. Furthermore, it would be desirable to have a method to obtain such drug substances in a dry powder state. The dry state offers the possibility to develop other dosage forms, for example, dry powder formulations for pulmonary delivery and tablets for oral or sublingual administration. The discussion that follows discloses pharmaceutical compositions containing natural cannabinoid compounds like THC that help to fulfill these needs.

### SUMMARY OF THE INVENTION

It has now surprisingly been found that highly lipophilic compounds like natural cannabinoid compounds can be stabilized against oxidation and isomerization by the incorporation in sugar glasses or sugar alcohol glasses by the mechanism mentioned herein. Furthermore, it was found that the sugar glass technology also leads to improved bioavailability of these compounds. As the natural cannabinoid compounds are incorporated monomolecularly, the dissolution rate of these compounds will be determined by the dissolution rate of the sugar glass. Because the dissolution rate of the sugar glass is much higher than that of the natural cannabinoid compound, the drug will be presented to the absorbing membrane more rapidly.

### BRIEF DESCRIPTION OF THE DRAWINGS

Other advantages of the present invention will be readily appreciated as the same becomes better understood by reference to the following detailed description when considered in connection with the accompanying drawing wherein:

FIG. 1 is a line chart illustrating the glass transition of inulin at different humidities.

FIG. 2 is a line graph illustrating the water sorption isotherm of freeze fried inulin.

FIG. 3 is a line graph illustrating the stabilization of a product containing 4.00% THC, freeze dried from water-TBA solution.

FIG. 4 is a line graph illustrating the stabilization of a product containing 3.34% THC, spray dried from water-1-propanol solution.

FIG. 5 is a line graph illustrating the stabilization of a product containing 7.77% THC, spray dried from water-1-propanol solution.

FIG. 6 is a line graph illustrating the stabilization of a product containing 4.00% THC, spray dried from water-ethanol solution.

FIG. 7 is a line graph illustrating the fraction of nondegraded THC over time in a physical mixture (4.00%).

FIG. 8 is a line graph illustrating the fraction of nondegraded THC over time of pure THC.

### DETAILED DESCRIPTION

While the present invention may be embodied in many different forms, several specific embodiments are discussed herein with the understanding that the present disclosure is to be considered only as an exemplification of the principles of the invention, and it is not intended to limit the invention to the embodiments illustrated.

In a first embodiment of the present invention, a pharmaceutical composition comprises a natural cannabinoid compound and a glass of a sugar or sugar alcohol or a mixture of sugars or sugar alcohols, and is characterized in that the natural cannabinoid compound is incorporated in the sugar as a monomolecular encapsulation without formation of a guest-host complex. The compound is incorporated in the sugar glass when there is a monomolecular inclusion of substantially every cannabinoid molecule in the sugar matrix. Therefore the formed delivery system according to this embodiment of the invention can be regarded as a monophasic delivery system. The natural cannabinoid molecules are randomly orientated within the sugar glass. In contrast to guest-host complexes like complexes with cyclodextrins, for example, once dissolved there remains no interaction between the cannabinoid compounds and the dissolved sugar molecules.

Incorporation of a cannabinoid compound in the sugar glass will result in a decrease of the glass transition temperature (Tg) of the sugar glass, a substantial disappearance of the Tg of the cannabinoid compound, and an increased dissolution rate of the cannabinoid compound. Furthermore, scanning electron microscopy can indicate whether the compound is incorporated. In one embodiment of the present invention, the natural cannabinoid compound is THC.

In another embodiment of the present invention, to obtain high stability of the formulation, the sugar glass has a glass transition temperature (Tg) of above about 50° C. at normal environmental conditions. Such conditions result in a low tendency to crystallize. Normal environmental conditions are defined as about 20 to 25° C. and up to about 40% relative humidity.

In the framework of the present invention the expression “natural cannabinoid compound” includes non-natural derivatives of cannabinoids which can be obtained by derivatization of natural cannabinoids and which are unstable like natural cannabinoids.

In the framework of the present invention the expression sugar includes polysugars and the expression sugar alcohols includes poly sugar alcohols. In one embodiment of the present invention, the sugar is a non-reducing sugar. A non reducing sugar is a sugar, which does not have or cannot form reactive aldehyde or ketone groups. Examples of non-reducing sugars are trehalose and fructanes such as inulines.

In another embodiment of the present invention, non-reducing sugars useful in the present invention are fructans or mixtures thereof. A fructan is generally understood to mean any oligo- or polysaccharide which contains a plurality of anhydrofructan units. The fructans can have a polydisperse chain length distribution, and can have a straight or branched chain. In one embodiment, the fructans contain mainly β-1,2 bonds, as in inulin, but they can also contain β-2,6 bonds, as in levan. Suitable fructans can originate directly from a natural source, but may also have undergone modification. Examples of modifications are reactions known per se that lead to a lengthening or shortening of the chain length. In addition to naturally occurring polysaccharides, also industrially prepared polysaccharides, such as hydrolysis products which have shortened chains and fractionated products having a modified chain length are suitable in the present invention. A hydrolysis reaction to obtain a fructan having a reduced chain length can be carried out enzymatically (for instance with endoinulase), chemically (for instance with aqueous acid, physically (for instance thermally) or by the use of heterogeneous catalysis (for instance with an acid ion exchanger). Fractionation of fructans, such as inulin, can be achieved, for example, through crystallization at low temperature, separation with column chromatography, membrane filtration and selective precipitation with an alcohol. Other fructans, such as long-chain fructans, can be obtained, for instance through crystallization, from fructans from which mono-and disaccharides have been removed. Fructans whose chain length has been enzymatically extended can also serve as fructan in the present invention. Further, reduced fructans can be used, which are fructans whose reducing end groups, normally fructose groups, have been reduced, for instance with sodium borohydride, or hydrogen in the presence of a transition metal catalysts. Fructans which have been chemically modified, such as crosslinked fructans and hydroxyalkylated fructans, can also be used. The average chain length in all these fructans is expressed as the number-average degree of polymerization (DP). The abbreviation DP is defined as the average number of sugar units in the oligo- or polymer.

In yet another embodiment of the present invention, a reducing sugar are inulins or mixtures of inulins. Inulins are oligo- and polysaccharides, consisting β-1,2 bound fructose units with an α-D-glucopyranose unit at the reducing end of the molecule and are available with different degrees of polymerization (DP). In one embodiment, the inulins are inulins with a DP of greater than about 6 or a mixtures of inulins wherein each inulin has a DP of greater than about 6. In yet another embodiment, inulins or mixtures of inulins with a DP of between about 10 and about 30. In still another embodiment, inulins or mixtures of inulins with a DP of between about 15 and about 25. Inulin occur, for example, in the roots and tubers of plants of the Liliaceae and Compositae families. An important source for the production of inulin is the Jerusalem artichoke, the dahlia and the chicory root. Industrial production starts mainly from the chicory root. The main difference between inulins originating from the different natural sources resides in the degree of polymerization (DP), which can vary from about 6 in Jerusalem artichokes to 10-14 in chicory roots and higher than 20 in the dahlia. Inulin is an oligo- or polysaccharide which in amorphous condition has favorable physicochemical properties for the application as auxiliary substance in pharmaceutical formulations. These physicochemical properties are: (adjustable) high glass transition temperature, no reducing aldehyde groups and normally a low rate of crystallization. Further inulin is non toxic and inexpensive.

In one embodiment of the present invention, the weight ratio of natural cannabinoid compound to sugar or sugar alcohol is in the range of between about 1:5 to about 1:100, and in yet another embodiment, the range is between about 1:10 and about 1:50, and in yet another embodiment, the range between about 1:12 and about 1:25.

The pharmaceutical composition according to the present invention may be further processed into a tablet such as a normal oral tablet, a sublingual tablet, a buccal tablet or an orally disintegrating or dissolving tablet, a capsule, a lozenge, an enema, a suppository, a product for transdermal administration, a powder for pulmonary administration, or a rod or suspension for subcutaneous or intramuscular administration. These forms of administration are known in the art and the person skilled in the art will be capable to process the composition according to the present invention into the desired form of administration. In one embodiment of the present invention, a formulation are those intended for oral administration or pulmonary administration.

In one embodiment of the present invention, the preparation of sugar glasses according to the present invention is by freeze drying. Also other drying techniques such as spray drying, vacuum drying, and super critical drying can be employed. The first step to prepare sugar glasses with incorporated natural cannabinoid compounds by means of these techniques is to make a solution in which both substances are dissolved. However, due to the hydrophilic nature of sugars and the lipophilic nature of the natural cannabinoid compounds, these compounds are hard to dissolve in the same solvent. It has now been found that this problem can be solved by the application of mixtures of solvents. Water is a good solvent for sugars and sugar alcohols, whereas various organic solvents such as alcohols are good solvents for natural cannabinoid compounds. Since water and alcohols mix very well it is likely that at a certain water/alcohol ratio both substances will dissolve to a certain extent.

Therefore the present invention is also related to a method of preparation of a pharmaceutical composition comprising a natural cannabinoid compound and a glass of a sugar or a mixture of sugars wherein the natural cannabinoid compound is incorporated in the sugar glass as a monomolecular encapsulation without formation of a guest-host complex, characterized in that

a) the natural cannabinoid compound is dissolved in an organic solvent that is soluble in water and the sugar or mixture of sugars is dissolved in water;

b) the dissolved cannabinoid compound and the dissolved sugar or mixture of sugars are mixed in such a way that a sufficiently stable mixture is obtained;

c) the mixture is freeze dried, spray dried, vacuum dried, or super critical dried.

Organic solvents which are suitable to form a stable mixture with the sugar, water and the natural cannabinoid compound are solvents which are mixable with water such as dimethylsulfoxide (DMSO), N,N-dimethylformamide (DMF), acetonitrile, ethylacetate and lower alcohols. As the solvents have to be removed by spray drying or freeze drying the solvents in one embodiment of the present invention have a reasonable vapor pressure at the drying temperature. Therefore, in one embodiment of the present invention the lower alcohols are defined as C₁-C₆ alcohols, wherein the alkyl chain may be branched or unbranched. In yet another embodiment of the present invention, the alcohols are C₂-C₄ alcohols such as ethanol, n-propyl alcohol and t-butyl alcohol. In still another embodiment, the solvent is t-butyl alcohol.

The ratios between the cannabinoid compound, the solvent, water and the sugar or mixture of sugars should be chosen in such a way that a sufficiently stable solution is obtained. Optionally a surfactant can be added to improve the stability. A solution is judged as sufficiently stable if no clouding appears in the solution within the time of processing, for example, within 120 minutes, 60 minutes, 30 minutes or 10 minutes. For a spray drying process a typical time of processing is 30 minutes. For a freeze drying process the solution should be clear until it is frozen. A typical time of processing here is 10 minutes.

In one embodiment of the present invention, the amount of water after the drying process is below about 3%. In one embodiment of the present invention, the amount of solvent is below 3%. It will be clear for a person skilled in the art that the time required for drying can be derived from parameters like sample thickness, sample temperature, pressure, and condenser temperature.

Although the use of a spray drying process for the preparation of sugar glasses of cannabinoid compounds leads to a significant improvement of the stability of the compounds, the best results are obtained with a freeze drying process. Therefore, in one embodiment, the method of drying in the present invention is freeze drying. In the first phase of the freeze drying process the solution is frozen. This first phase should preferably be performed rapidly and should reduce the sample temperature to below Tg′, which is the temperature of the freeze concentrated fraction (see D. L. Teagarden, Eur. J. Pharm. Sci., 15, 115-133, 2002). Freeze drying below the Tg′ results in a porous cake, while a collapsed cake is obtained above the Tg′. In one embodiment of the present invention, a porous cake is used because it can be processed more easily into, for example, a powder for tableting or formulations for pulmonary delivery. Moreover, freeze drying above the Tg′ may lead to crystallization of the sugar. This will prevent the incorporation of the drug in a glass and as a result reduced stabilization will be achieved.

The methods, kits, combinations, and compositions of the present invention provide enhanced treatment options for treating any condition or disorder responsive to cannabinoids in a subject in need thereof as compared to those currently available. The methods, kits, combinations, and compositions of the present invention are useful in treatment and prevention of a very wide range of disorders, including, for example, nausea, vomiting, anorexia, cachexia, pain, gastrointestinal tract distress (such as heartburn, indigestion, stomachache, sour stomach), inflammatory bowel disease, Crohn's disease, gastritis, irritable bowel syndrome, ulcerative colitis, migraine headaches, postmenstrual syndrome, Alzheimer's dementia, agitation, muscle spasms and other involuntary movement disorders, Parkinson's disease and Parkinsonian-type symptoms, glaucoma, and anxiety disorders (hereafter collectively, “disorder” or “disorders”).

Besides being useful for human treatment of such disorders, the present invention is also useful for veterinary treatment of companion mammals, exotic animals, and farm animals, including mammals, birds, rodents, and the like. More particularly, the methods, kits, combinations, and compositions of the present invention are useful for treatment of a disorder in a horse, cow, chicken, pig, dog, or cat.

The present invention includes methods, kits, combinations, and compositions for reversing, halting, or slowing the progression of a disorder once it becomes clinically evident, or treating the symptoms associated with or related to a disorder. The subject may already have a disorder at the time of administration, or be at risk of developing a disorder.

A therapeutic agent (or the therapeutic agents) of the present invention is used in a method, kit, combination, and/or composition in a “disorder-effective amount.” A “disorder-effective amount” is intended to qualify the amount of an agent (or agents) required to treat or prevent a disorder in a subject, or relieve to some extent one or more of the symptoms associated with, or related to, a disorder in a subject. In a mammal, this includes, but is not limited to, improving or alleviating the above stated diseases.

The term “prevent” or “prevention,” in relation to a disorder, means no disorder, condition, or disease development if none had occurred, or no further disorder, condition, or disease development if there had already been development of a disorder, condition, or disease.

When the compositions of the present invention are used in a “disorder-effective amount” this means that the dose of the therapeutic agent (or agents) is such that a therapeutic level of agent is delivered to the bloodstream over the term that the composition is to be used. Such delivery is dependent on a number of variables including the time period for which the individual dosage unit is to be used, or the flux rate of the therapeutic agent into the systemic circulation of the subject. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of a disorder in accordance with the present invention. In terms of treatment protocols, it should be appreciated that the dosage to be administered will depend on several factors, including the particular agent that is administered, the route administered, the physical state of the particular agent, the condition of the particular subject, etc.

Toxicity and therapeutic efficacy of the therapeutic agents (and hence the dosing) of the inventive compositions can be determined by standard pharmaceutical procedures, for example, for determining LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. In one embodiment of the present invention, compounds that exhibit large therapeutic indices are used. While compounds that exhibit toxic side effects may be used, care should be taken to design a delivery system that targets such compounds to the site of affected tissue in order to minimize potential damage to uninfected cells and, thereby, reduce side effects.

The compositions of the present invention can be administered by any conventional means available for use in conjunction with pharmaceuticals, either as individual therapeutic compounds or as a combination of therapeutic compounds. The compositions of the present invention include those suitable for oral or buccal, sublingual, nasal, pulmonary or rectal administration, although the most suitable route in any given case will depend on the nature and severity of the condition being treated and on the nature of the particular compound which is being used.

The pharmaceutical compositions of the present invention can be administered for treating, preventing, or reducing the risk of developing a disorder in a subject by any means that produce contact of these compounds with their site of action in the body, For example, in the lungs, gastrointestinal fluid or tract of a subject, including the stomach and/or the small intestine, or in the ileum, blood, brains, kidneys, spleen and/or liver of a subject.

The pharmaceutical compositions of the present invention can be administered in dosage forms containing conventional nontoxic pharmaceutically acceptable (functional) excipients such as fillers, binders, carriers, adjuvants, and vehicles as desired. The carrier materials that can be employed in making the immediate-release or controlled-release components of the present invention are any of those commonly used excipients in pharmaceutics and should be selected on the basis of compatibility with the natural cannabinoid pharmaceutical agent and the release profile properties of the desired dosage form. Illustratively, a pharmaceutical excipient except active drugs are chosen below as examples:

(a) Binders such as acacia, alginic acid and salts thereof, cellulose derivatives, methylcellulose, hydroxyethyl cellulose, hydroxypropyl cellulose, magnesium aluminum silicate, polyethylene glycol, gums, polysaccharide acids, bentonites, hydroxypropyl methylcellulose, gelatin, polyvinylpyrrolidone, polyvinylpyrrolidone/vinyl acetate copolymer, crospovidone, povidone, polymethacrylates, hydroxypropylmethylcellulose, hydroxypropylcellulose, starch, pregelatinized starch, ethylcellulose, tragacanth, dextrin, microcrystalline cellulose, sucrose, or glucose, and the like.

(b) Disintegration agents such as starches, pregelatinized corn starch, pregelatinized starch, celluloses, cross-linked carboxymethylcellulose, crospovidone, cross-linked polyvinylpyrrolidone, a calcium, a sodium alginate complex, clays, alginates, gums, or sodium starch glycolate, and any disintegration agents used in tablet preparations.

(c) Filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

(d) Surfactants such as sodium lauryl sulfate, sorbitan monooleate, polyoxyethylene sorbitan monooleate, polysorbates, polaxomers, bile salts, glyceryl monostearate, Pluronic™ line (BASF), and the like.

(e) pH correcting agents (buffers) such as citric acid, succinic acid, fumaric acid, malic acid, tartaric acid, maleic acid, glutaric acid sodium bicarbonate and sodium carbonate and the like.

(f) Stabilizers such as any antioxidation agents, buffers, or acids, and the like, can also be utilized.

(g) Lubricants such as magnesium stearate, calcium hydroxide, talc, sodium stearyl fumarate, hydrogenated vegetable oil, stearic acid, glyceryl behapate, magnesium, calcium and sodium stearates, stearic acid, talc, waxes, Stearowet, boric acid, sodium benzoate, sodium acetate, sodium chloride, DL-leucine, polyethylene glycols, sodium oleate, or sodium lauryl sulfate, and the like.

(h) Wetting agents such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, or sodium lauryl sulfate, and the like.

(i) Diluents such lactose, starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, or bentonite, and the like.

(j) Anti-adherents or glidants such as talc, corn starch, DL-leucine, sodium lauryl sulfate, and magnesium, calcium, or sodium stearates, and the like.

(k) Pharmaceutically compatible carrier comprises acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, or pregelatinized starch, and the like.

Additionally, drug formulations are discussed in, for example, Remington's, The Science and Practice of Pharmacy (2000); Lieberman, H. A. and Lachman, L., Eds., Pharmaceutical Dosage Forms, Marcel Decker, New York, N.Y., 1980; and Liebeman et al., Pharmaceutical Dosage Forms (Volumes 1-3, 1990).

In making the compositions of the present invention, some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, microcrystalline cellulose, methyl cellulose, hydroxypropylcellulose (HPC) and cross-linked polyvinyl pyrrolidone. The formulations can additionally include: lubricating agents, such as talc, magnesium stearate and mineral oil; wetting agents; emulsifying and suspending agents; preserving agents, such as methyl- and propylhydroxybenzoates; sweetening agents; and flavoring agents. For purposes of this application, excipients can serve the function as carriers and vice versa.

When the excipient serves as a diluent, it can be a solid, semi-solid or liquid material, which acts as a vehicle, carrier or medium for the active ingredient. Thus, the compositions can be in the form of a tablet, pill, powder, lozenge, sachet, cachet, troche, suspension, emulsion, aerosol (as a solid or in a liquid medium), soft and hard gelatin capsule, sterile packaged powder, dispensable powder, granule, or liquid.

Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents and pharmaceutically compatible carriers. In one embodiment of the present invention, the manufacturing processes may employ one or a combination of methods: (1) dry mixing, (2) direct compression, (3) milling, (4) dry or non-aqueous granulation, (5) melt granulation, or (6) fusion. Lachman et al., The Theory and Practice of Industrial Pharmacy (1986). Such tablets may also comprise film coatings, which disintegrate upon oral ingestion or upon contact with diluent.

Compressed tablets are solid dosage forms prepared by compacting a formulation containing an acid-labile pharmaceutical agent and/or buffering agent and/or excipient selected to aid the processing and improve the properties of the product. The term “compressed tablet” generally refers to a plain, uncoated tablet for oral ingestion, prepared by a single compression or by pre-compaction tapping followed by a final compression.

The tablets or pills of the present invention may be coated or otherwise compounded to provide a dosage form affording the advantage of improved handling or storage characteristics. For example, the tablet or pill can comprise an inner dosage and an outer dosage component, the latter being in the form of an envelope over the former.

Since a tablet may be used to form rapidly disintegrating tablets, chewable tablets, lozenges, troches or swallowable tablets; the intermediate formulations, as well as the process for preparing them, provide additional aspects of the present invention.

Effervescent tablets and powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid. When the salts are added to water, the acids and the base react to liberate carbon dioxide gas, thereby causing “effervescence.”

The liquid forms in which the novel compositions of the present invention may be incorporated for administration orally include non-aqueous solutions; suitably flavored non aqueous syrups; oil suspensions; and flavored emulsions with edible oils, such as cottonseed oil, sesame oil, coconut oil or peanut oil, as well as elixirs and similar pharmaceutical vehicles.

Many other types of release delivery systems are available and known to those of ordinary skill in the art. They include polymer-based systems, such as polylactic and polyglycolic acid, polyanhydrides and polycaprolactone; nonpolymer systems that are lipids, including sterols, such as cholesterol, cholesterol esters and fatty acids, or neutral fats, such as mono-, di- and triglycerides; hydrogel release systems; silastic systems; peptide-based systems; wax coatings; compressed tablets using conventional binders (See, for example, Lieberman et al., Pharmaceutical Dosage Forms, 2 Ed., Vol. 1, pp. 209-214 (1990), and excipients; partially fused implants; and the like. Specific examples include, but are not limited to: (a) erosional systems in which the polysaccharide is contained in a form within a matrix, found in U.S. Pat. Nos. 4,452,775; 4,667,014; and 4,748,034 and 5,239,660; and (b) diffusional systems in which an active component permeates at a controlled rate through a polymer, found in U.S. Pat. Nos. 3,832,253 and 3,854,480.

Additionally, the methods, kits, combinations, and compositions of the present invention optionally include a salt, an ester, an amide, an enantiomer, an isomer, a tautomer, a prodrug, or a derivative of an active agent of the present invention. Certain compounds of the present invention may exist in different isomeric (for example, enantiomers and diastereoisomers) forms. The invention contemplates all such isomers both in pure form and in admixture, including racemic mixtures. Enol forms are also included.

The term “derivative” refers to a compound that is produced from another compound of similar structure by the replacement of substitution of one atom, molecule or group by another. For example, a hydrogen atom of a compound may be substituted by alkyl, acyl, amino, hydroxyl, halo, haloalkyl, etc., to produce a derivative of that compound.

Certain natural cannabinoid compounds of the invention also form pharmaceutically acceptable salts, for example, acid addition salts. For example, the nitrogen atoms may form salts with acids. Examples of suitable acids for salt formation are hydrochloric, sulfuric, phosphoric, acetic, citric, oxalic, malonic, salicylic, malic, fumaric, succinic, ascorbic, maleic, methanesulfonic, and other mineral and carboxylic acids well known to those in the art. The salts are prepared by contacting the free base form with a sufficient amount of the desired acid to produce a salt in the conventional manner. The free base forms may be regenerated by treating the salt with a suitable dilute aqueous base solution such as dilute aqueous hydroxide, potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. See, for example, S. M. Berge, et al., “Pharmaceutical Salts,” J. Pharm. Sci., 66: 1-19 (1977).

Individual enantiomeric forms of compounds of the present invention can be separated from mixtures thereof by techniques well known in the art. For example, a mixture of diastereoisomeric salts may be formed by reacting the compounds of the present invention with an optically pure form of the acid, followed by purification of the mixture of diastereoisomers by recrystallization or chromatography and subsequent recovery of the resolved compound from the salt by basification. Alternatively, the optical isomers of the compounds of the present invention can be separated from one another by chromatographic techniques employing separation on an optically active chromatographic medium.

The present methods, kits, and compositions can also be used in combination (“combination therapy”) with another pharmaceutical agent that is indicated for treating or preventing a disorder, such as, for example, opioid or opiate analgesics, NSAIDs, COX-2 inhibitors, and anti-emetics (for example, ondansetron). These drugs have certain disadvantages associated with their use. Some of these drugs are not completely effective in the treatment of the aforementioned conditions and/or produce adverse side effects, such as mental confusion, constipation, respiratory depression, and diarrhea. However, when used in conjunction with the present invention, that is, in combination therapy, many if not all of these unwanted side effects can be reduced or eliminated. The reduced side effect profile of these drugs is generally attributed to, for example, the reduce dosage necessary to achieve a therapeutic effect with the administered combination.

The phrase “combination therapy” embraces the administration of a composition of the present invention in conjunction with another pharmaceutical agent that is indicated for treating or preventing a disorder in a subject, as part of a specific treatment regimen intended to provide a beneficial effect from the co-action of these therapeutic agents for the treatment of a disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. The composition of the present invention can be administered orally or nasogastric, while the other therapeutic agent of the combination can be administered by any appropriate route for that particular agent, including, but not limited to, an oral route, inhalation, a percutaneous route, an intravenous route, an intramuscular route, or by direct absorption through mucous membrane tissues. For example, the composition of the present invention is administered orally or nasogastric and the therapeutic agent of the combination may be administered orally, or percutaneously. The sequence in which the therapeutic agents are administered is not narrowly critical. “Combination therapy” also can embrace the administration of the therapeutic agents as described above in further combination with other biologically active ingredients, such as, but not limited to, a pain reliever, such as a steroidal or nonsteroidal antiinflammatory drug, or an agent for improving stomach motility, for example, and with non-drug therapies, such as, but not limited to, surgery.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation, rectally, topically, buccally, sublingually, or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

In another embodiment of the present invention, the composition of the present invention comes in the form of a kit or package containing one or more of the compositions or therapeutic agents of the present invention. The composition containing the composition or therapeutic agent can be packaged in the form of a kit or package in which hourly, daily, weekly, or monthly (or other periodic) dosages are arranged for proper sequential or simultaneous administration. The present invention further provides a kit or package containing a plurality of dosage units, adapted for successive daily administration, each dosage unit comprising at least one of the compositions or therapeutic agents of the present invention. This drug delivery system can be used to facilitate administration of any of the various embodiments of the compositions and therapeutic agents of the present invention. In one embodiment, the system contains a plurality of doses to be to be administered daily or as needed for symptomatic relief. The kit or package can also contain agents utilized in combination therapy to facilitate proper administration of the dosage forms. The kit or package can also contain a set of instructions for the subject.

The use of the term “about” in the present disclosure means “approximately,” and use of the term “about” indicates that dosages and amounts outside that cited may also be effective and safe, and such dosages and amounts are also encompassed by the scope of the present claims.

The following example is only intended to further illustrate the invention, in more detail, and therefore this example is not deemed to restrict the scope of the invention in any way.

### EXAMPLES

### Example 1

Preparation and Properties of Inulin Glasses of Δ⁹-Tetrahydrocannabinol.

Materials

Inulin, type TEX!803, was provided by Sensus, Roosendaal, The Netherlands. Purified Δ⁹-tetrahydrocannabinol (THC) was a gift of Unimed. All other chemicals were of reagent or analytical grade and purchased from commercial suppliers.

Methods

Physico-Chemical Characterization of Inulin

Determination of the Degree of Polymerisation of Inulin

The average degree of polymerisation (DP) of inulin was determined as follows: an inulin solution was acidified to a pH of 1.45 by adding 3 N HCl. Subsequently, the temperature was raised to 80° C. by which the inulin was degraded to fructose and glucose. After cooling to room temperature, the pH was adjusted to 6-8 by adding 1.5 M NaOH. The fructose/glucose ratio was determined by means of HPLC. An Aminex HPX-87C column was used. Samples were eluated with MilliQ-water of 80° C. at a flow rate of 0.6 mL/min. An IR detector was used to measure the amounts of fructose and glucose. The DP is the ratio of the fructose content and the glucose content plus one.

Determination of the Number of Reducing Groups

The number of reducing groups was determined by means of the Summer-assay according to the following procedure. A solution of 20 g NaK-tartrate tetrahydrate, 1 g dinitrosalicylic acid, 1 g NaOH, and 200 mg phenol in 100 mL water was prepared. To 1.5 ml of this solution, 1.0 mL of an aqueous solution containing the sugar to be analysed was added. Subsequently, 100 μL of a freshly prepared solution of 0.24 M of Na₂SO₃ in water was added to this mixture. The resulting mixture was vortexed and then placed in a waterbath of 95° C. After 15 min, the samples were removed from the waterbath and allowed to cool to room temperature. The extinction of the samples was measured at 620 nm. The calibration curve was made using aqueous solutions with a glucose concentration of 0.10-1.00 mg/mL. Measurements were performed in triplicate.

Differential Scanning Calorimetry (DSC)

The glass transition temperature (Tg) of freeze dried inulin equilibrated at 0%, 45% and 60% RH was determined by modulated DSC (DSC 2920 differential scanning calorimeter, TA instruments, Gent, Belgium). A modulation amplitude of ±0.318° C. every 60 sec and a heating rate of 2° C./min was used. During measurement, the sample cell was purged with nitrogen at a flow rate of 35 mL/min. The midpoint of the deflection in the reversing heat flow versus temperature curve was taken as the Tg. The Tg was determined in duplicate.

The glass transition temperature of the freeze concentrated fraction (Tg′) of a 9.6% w/v solution of inulin in 60/40 v/v water/t-butyl alcohol mixture was measured by means of conventional DSC. Solutions were cooled to −70° C. with a cooling rate of 10° C./min. Subsequently, the samples were heated to 40° C. with a rate of 2° C./min. During these measurements, the sample cell was purged with helium at a flow rate of 35 mL/min. The midpoint of the deflection in the heat flow versus temperature curve was taken as the Tg′. The Tg′ was determined in duplicate.

Physical Stability Amorphous Inulin

To evaluate the physical stability of amorphous inulin, porous cakes of amorphous inulin obtained by means of freeze drying were humidified at 20° C. by transferring them into climate chambers conditioned at 45% or 60% RH respectively. After equilibration, the samples were judged visually whether they remained unchanged or were collapsed.

Dynamic Vapour Sorption

Water sorption isotherm of freeze dried inulin was measured at ambient pressures and 25° C. using a gravimetric sorption analyser (DVS-1000 Water Sorption Instrument, Surface Measurement Systems Limited, London, UK). The uptake of water by inulin was measured from 0% to 90% RH with steps of 10% RH. The initial sample weight was about 10 mg. It was assumed that equilibrium was reached when the change of weight was less than 0.9 μg during a ten minutes period.

Physico-Chemical Characterization of THC

Solubility in Water

Pure water was added to an excess of THC. The resulting dispersion was stirred at 20° C. using a magnetic stirrer. After 3 days the dispersion was centrifuged and the concentration of THC in the supernatant was determined spectrophotometrically at a wavelength of 210 nm. The sample was diluted with ethanol. A calibration curve was established using solutions of THC in ethanol of known concentrations (1.244-12.44 μg/mL).

Dynamic Vapour Sorption

The water sorption of THC was determined according to the procedure described above for inulin. The THC was dissolved in methanol before it was put in the DVS-1000 instrument. During initial exposure to a dry nitrogen flow, the methanol was evaporated. As soon as about 90% of the solvent was evaporated, additional THC solution was added to the sample-cup. This procedure was repeated until 15 mg of pure THC was present in the sample cup. After the evaporation of the last methanol, the relative humidity was increased from 0% to 90% in steps of 10%.

Differential Scanning Calorimetry (DSC)

The thermal behavior of THC was determined by mDSC. A modulation amplitude of ±0.318° C. every 60 sec and a heating rate of 2° C./min was used. During measurement, the sample cell was purged with nitrogen at a flow rate of 35 mL/min. A blob of pure THC was put in the sample cup. After initial cooling the sample was first scanned till 50° C. In this way the blob was able to spread over the entire bottom of the sample pan, thereby increasing the surface available for heat transfer during the second scan. The sample was then cooled until −40° C. and heated to 350° C.

Production of THC Containing Samples

Preparation of Solutions for Spray Drying or Freeze Drying

Three different formulations were prepared for spray drying and one for freeze drying (Table 2). Formulations 5, 6, 9 and 12 were prepared by dissolving, separately inulin in water and THC in the appropriate alcohol.

The suitable volume ratio of water/alcohol was investigated by testing the stability of 10% w/v inulin solutions with different ratio's water/alcohol. Inulin was dissolved in different amounts of water (3 to 7 mL). Subsequently different amounts of alcohol were added until a total volume of 10 mL. For THC the same procedure was followed, but now water was added to the alcoholic THC solution. The solution was judged as sufficiently stable if no clouding appeared within the time of processing. For spray drying batches were made which required up to half an hour of spraying. Therefore the solution should be clear for at least that period of time. For freeze drying the solution should be clear until it is frozen. In this case ten minutes is sufficient. Furthermore, it was investigated whether the aqueous inulin solution could be added slowly or should be mixed instantaneously.

TABLE 2
Formulations for spray drying and freeze drying

| | | | [Inulin] | THC/ |
|---|---|---|---|---|
| Formu- | Drying | | (mg/ | Inulin |
| lation | method | Solvent | mL) | (m %) |
| 9 | Spray drying | H₂O/EtOH = 50/50(v/v) | 47.73 | 4.00% |
| 5 | Spray drying | H₂O/1-PrOH = 60/40(v/v) | 49.00 | 3.34% |
| 6 | Spray drying | H₂O/1-PrOH = 60/40(v/v) | 46.17 | 7.77% |
| 12 | Freeze drying | H₂O/t-BuOH = 60/40(v/v) | 96.00 | 4.00% |

Spray Drying

Spray drying was performed using a Büchi 190 mini spray dryer (Büchi, Flawil, Switzerland). Typical operating conditions were according to the following settings: nitrogen-gas inlet temperature: 148° C. which gave an outlet temperature of 87° C., drying air flow 525 L/h, aspirator flow setting: 20, and pump control setting: 6. After spray drying, the formed powder was collected in a 50 mL bottle and flushed with nitrogen for about 15 minutes. The product was stored at −18° C.

Freeze Drying

Freeze drying was performed using a Christ model Alpha 2-4 lyophilizer (Salm en Kipp, Breukelen, The Netherlands). In a typical experiment, 20 mL glass vials were charged with 2-5 mL solution. The solutions were frozen in liquid nitrogen and subsequently lyophilized at shelf temperature of −30° C., a condenser temperature of −53° C., and a pressure of 0.220 mbar for 1-3 days. Subsequently, the shelf temperature was gradually raised to 20° C. and pressure was gradually decreased to 0.05 mBar during 6 hours. The samples were stored in a vacuum desiccator for at least one day.

Stability Study of THC Containing Samples

Samples were stored under five different conditions; given in Table 3. At different time intervals samples were taken and the amount of nondegraded THC was determined by means of HPLC. Pure THC and a physical mixture of THC and inulin were used as controls. Samples of pure THC were made as follows. 720.5 mg of THC was dissolved in 20.00 mL of methanol. 70 μL of this solution was transferred into a glass vial with a diameter of 24 mm. Subsequently the solvent was allowed to evaporate in a flow of dry nitrogen, leaving 2.52 mg of pure THC in the vial. A physical mixture was prepared by weighing about 192 mg of inulin into a vial with a diameter of 24 mm. Subsequently, 200 μL of a 36.025 mg/mL methanolic solution of THC was added, yielding a mixture containing 4.0% THC by mass.

TABLE 3
Storage conditions THC containing samples

| Temperature (° C.) | Relative humidity (%) | Atmosphere |
|---|---|---|
| 20 | 0 | low [O₂] |
| 20 | 45 | air |
| 20 | 60 | air |
| 47 | 0 | low [O₂] |
| 47 | 5 | air |

THC-Analysis

The samples were analysed by means of HPLC. They were prepared as follows. Methanol was added to samples. An ultrasonic treatment of ten minutes dispersed the product throughout the methanol. The suspension thus obtained, was shaken manually. After two days of extraction a sample was taken. The sample was centrifuged and the supernatant was diluted with methanol. In a control experiment, it was shown that ultrasonic treatment induced no degradation of THC. During the two days of extraction, no significant degradation of THC was measured. An ISCO model 2350 system equipped with a Photodiode Array UV-VIS Detector (Shimadzu SPD-M6A model) and a Chrompack Nucleosil 100 C18 column (4.6×250 mm) was used. Samples (20 μL) were injected with a Kontron Instruments HPLC 360 Autosampler and eluted with a mixture of methanol/water=86/14 (v/v). The flow rate was 1.5 mL/min. The absorbance was measured at 214 nm. The collected data were analysed using SPD-MXA software. In a chromatogram of untreated THC, a large peak was observed at a retention time of 7.5 min. In a chromatogram of THC which was intentionally partially degraded, the peak at a retention time of 7.5 min decreased in size while at shorter retention times new peaks appeared. The peak at a retention time of 7.5 min was ascribed to Δ⁹-THC. The other peaks were ascribed to degradation products. The content of (non-degraded) THC in processed samples was calculated from the area under the peak at an elution time of 7.5 min. A calibration curve was established using solutions of THC in methanol of known concentrations (0-122 μg/mL). In every HPLC-run some calibration points were included. The solutions used for this purpose showed no significant degradation during a period of 2 weeks at 4° C. Measurements were performed at least in duplicate.

Physico-Chemical Characterization of Inulin

The physico-chemical characteristics of the inulin used are summarized in Table 4.

TABLE 4
Physico-chemical characterization of inulin glasses

| | |
|---|---|
| Average degree of | 23 |
| polymerization | |
| % sugar units containing | 5.9 ± 0.1 |
| reducing groups | |
| Tg | 155.4 ± 0.1 ° C. |
| Tg′ | −24° C. |
| Physical stability at | Stable at RH ≦ 45%; collapsed at RH ≧ 60% |
| 20° C. | |
| Hygroscopicity | Change in mass = 0.22 * RH (%) + 0.61 |

A DP of inulin of 23 was found. For several reasons this value should be regarded as an indication. Inulin consists of linear β-D-(2→1) linked fructose oligomers ending with a α-D-(1→2) glucopyranose ring. Therefore, the DP can be calculated from the glucose/fructose ratio as presented here. However, commercially available inulins may contain inulin species of which the glucose endgroup is cleaved. The presence of these species will cause an overestimation of the DP. On the other hand commercially available inulins may also contain small amounts of glucose. The presence of these species will cause an underestimation of the DP.

Due to the specific linkages between the monosaccharide rings, inulin should contain no reducing groups. However, the Sumner assay showed that 5.9±0.1% of sugar units of the inulin used in this study contained reducing groups. The presence of reducing groups can be predominately ascribed to inulin species of which the glucose endgroup is cleaved although the presence of monosaccharides may have contributed too. These monosaccharides can be glucose and fructose. Fructose is a nonreducing sugar. However, during the Sumner assay, the sugar is subjected to a high temperature by which fructose can be easily converted into glucose (Lobry de Bruyn van Ekenstein rearrangement). Indeed in control experiments, it was found that fructose displayed one reducing group per molecule in the assay (data not shown). Therefore, the measured amount of reducing groups is probably overestimated.

A glass transition temperature (Tg) of inulin of 155.4±0.1° C. was found. This value is substantially higher than the Tgs of trehalose (120° C.) and sucrose (76° C.), sugars which are frequently used to stabilize unstable drugs. A high Tg is important because at temperatures above the Tg the material changes into the rubbery state. In the rubbery state the molecular mobility is strongly increased compared to glassy state, as a consequence the degradation rate of the enclosed drug substance is strongly increased. Besides that, also crystallization can occur in the rubbery state. During crystallization, the incorporated drug substance is expelled from the stabilizing matrix and the protection is completely lost. The Tgs may seem very high. However, sugar glasses absorb water upon exposure to humidified air (see below). Water acts as a plasticizer for sugar glasses and strongly decreases the Tg. Therefore, inulin glasses can absorb much more water than trehalose or sucrose glasses before the Tg is decreased to room temperature.

A Tg′ of inulin of −24° C. was found. Also this value is higher than the Tg's of trehalose (−36° C.) and sucrose (−39° C.). When freeze drying is chosen as the method of drying, it is preferrable that Tg′ is relatively high, because the sample temperature should remain below the Tg′. When the sample temperature is above Tg′, the freeze concentrated fraction is in the rubbery state and as mentioned above the molecular mobility is relatively high. Because concentration of the drug substance in the freeze concentrated fraction is very high, the degradation rate can be increased when compared to the starting solution. Furthermore, also in this case crystallization of the sugar may easily occur with concomitant deteriorating effects to the drug substance. Furthermore freeze drying below the Tg′ results in a porous cake, while a collapsed cake is obtained above the Tg′. In one embodiment of the present invention, a porous cake is used because it can be processed more easily into, for example, a powder for tableting or formulations for pulmonary delivery.

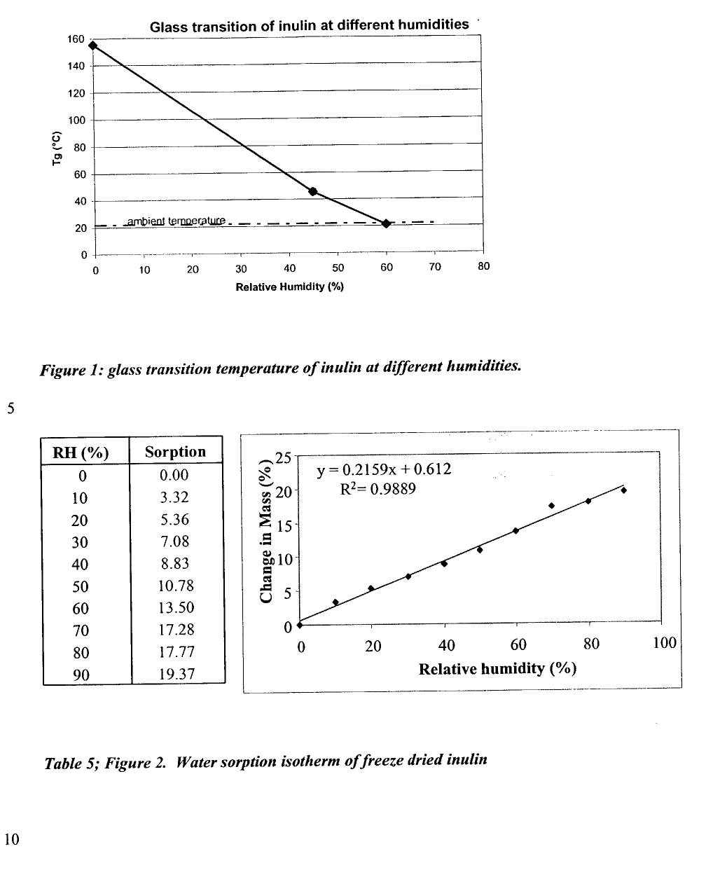

The physical stability of inulin glass at 20° C. was evaluated by exposing the glass to air of various relative humidities. It was found that porous cakes of inulin prepared by freeze drying remained unaffected up to an RH of 45%. At an RH of 60%, however, the porous cake collapsed. This means that at an RH between 45% and 60%, the sample absorbed water to such an extent that the Tg is passed. A short period of exposure to 60% RH may be applied to the freeze dried cake to have it partially collapsed. This partially collapsed material may form a suitable fast dissolving tablet with sufficient strength. The Tgs of freeze dried inulin after equilibration in 0, 45% and 60% RH are depicted in FIG. 1.

The moisture uptake of freeze dried inulin exposed to air of relative humidities ranging from 0 to 90% at 25° C. was measured using a gravimetric sorption analyser. Over the whole range of relative humidities, a linear relationship was found between the water uptake and the RH to which the sample was exposed (Table 5; FIG. 2). As found above, the Tg is passed at an RH between 45% and 60%. The linear relationship indicates that during the time frame of the experiment (hours) no crystallization of inulin takes place. When crystallization takes place and anhydrous crystals are formed the water content of the sample will drop to close to zero. On the other hand when crystals are formed which enclose water molecules, the water content of the sample remains more or less the same with increasing RH. These phenomena were observed with water sorption experiments with amorphous sugars like trehalose, sucrose, and lactose. Therefore, the results indicate that amorphous inulin crystallizes less easily than amorphous trehalose, sucrose, and lactose.

Physico-Chemical Characterization of THC

Solubility

The solubility of THC was found to be below 1 μg/mL (approximately 0.5 μg/mL).

Dynamic Vapour Sorption

Pure THC was found to absorb only 0.3% water after exposure to 90% RH. This extent of water uptake can probably be ascribed to adsorption onto rather than absorption into THC.

Differential Scanning Calorimetry

In the thermogram of THC a Tg of 10° C. was found. Furthermore an endothermic peak with an onset at 200° C. was found. From a thermodynamic point of view, it is expected that just above the Tg crystallization takes place. However, it is known that THC does not crystallize easily. As a consequence, at ambient temperature, THC is in the rubbery or liquid state. The endothermic peak is due to evaporation.

Production of THC Containing Samples

Water-Alkanol Solutions for Spray Drying or Freeze Drying

The three relevant alcohol's were added to a solution of inulin in water. It was determined for how long the obtained solution stayed clear. After 1 g of inulin was dissolved in 4 mL water, water and/or alcohol was added to a total volume of 10 mL, yielding a 10% w/v solution. The largest concentration of alcohol was thus obtained. THC was dissolved in the alcohol of interest. Subsequently, alcohol and/or water was added to give 0.4% w/v solutions. The compositions required to obtain a stable solution (defined in Materials and Methods) are given in table 5.

TABLE 5
Ratio's water-alcohol in solutions of inulin and THC.

| | % v/v water | alcohol |
|---|---|---|
| THC | 53% maximum | EtOH (ethanol) |
| | 62% maximum | n-PrOH (n-propanol) |
| | 63% maximum | TBA (t-butanol) |
| Inulin | 50% minimum | EtOH |
| | 60% minimum | n-PrOH |
| | 60% minimum | TBA |

Solutions for spray drying were prepared by adding the aqueous inulin solution to the THC solution. It turned out that this must be done quite fast, to prevent the inulin from clouding the mixture. The solutions stayed clear during the time necessary to spray the solution. The THC solution to be freeze dried was prepared by dissolving 690 mg THC in 20 mL TBA. Glass vials of 20 mL were each filled with 0.23 mL of the THC solution. Subsequently, the solutions were diluted with 0.57 mL of pure TBA. After that, 1.2 mL of an aqueous inulin solution (160 mg/mL) was added, the vials were shaken manually and frozen immediately afterwards.

Recovery of THC After Drying

The amount of THC in the spray dried samples, immediately after production, was lower than expected. Initially recoveries of about 50% were found. After changing both the atomizing gas flow and the gas flow from the heater to nitrogen, the recovery increased to 75%. In case of freeze drying, 100% of the expected amount of THC was found in the samples after the drying procedure.

Characterization of THC Containing Samples

Scanning Electron Microscopy

Scanning electron microscopy (SEM) photo's of the spray dried products showed the existence of agglomerates of small particles. These particles, having diameters of 1 to 5 μm, were hollow. The small size and the decreased density of the spray dried particles make them excellent for processing into dry powder formulations for inhalation. A SEM photo of a reference product (inulin without THC which was spray dried under the same conditions and with the same solvents) showed no differences. No THC spots are noticed on the particle surfaces of THC containing samples, indicating that THC is incorporated in the inulin matrix.

Stability of THC Containing Samples

The samples were exposed to conditions with O₂ or at low O₂ (indicated as nitrogen in the figures), at 20° C. and 47° C. respectively. Furthermore, they were exposed to two different humidities at 20° C., as summarized before. The spray dried products showed a slight change in color after they were collected from the spray-dryer.

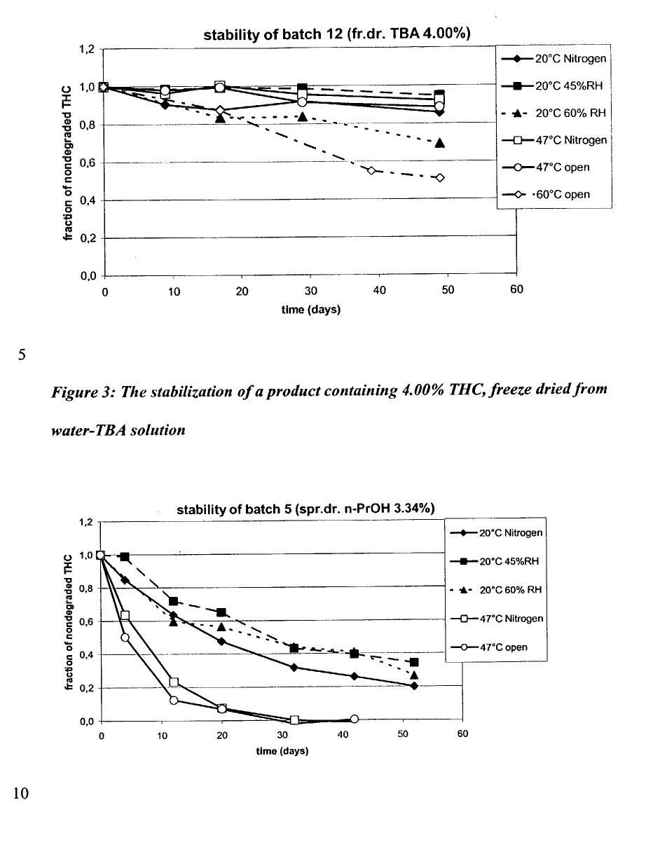

FIGS. 3-6 show the results of the batches 12, 5, 6, and 9. The amount of THC was determined. In the figures the fraction of Δ⁹-THC present in the samples after several exposure times is plotted for the five different climates.

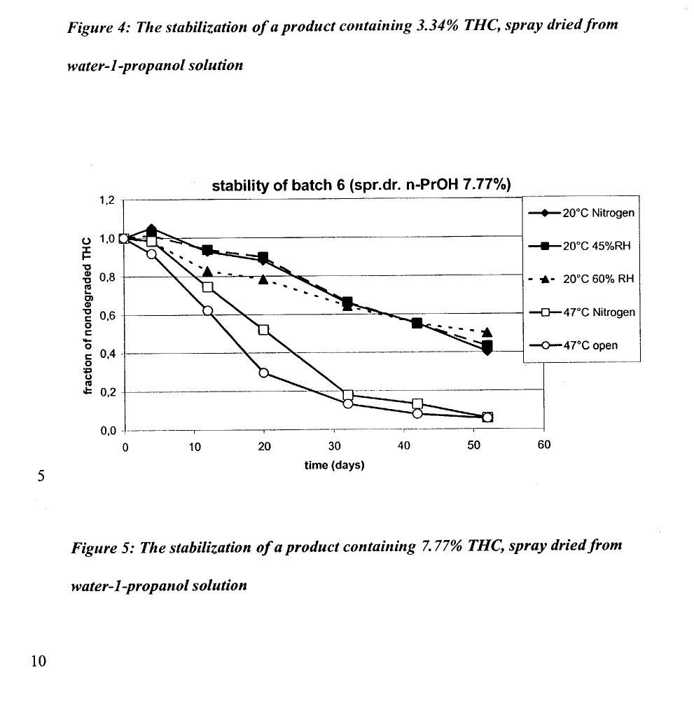

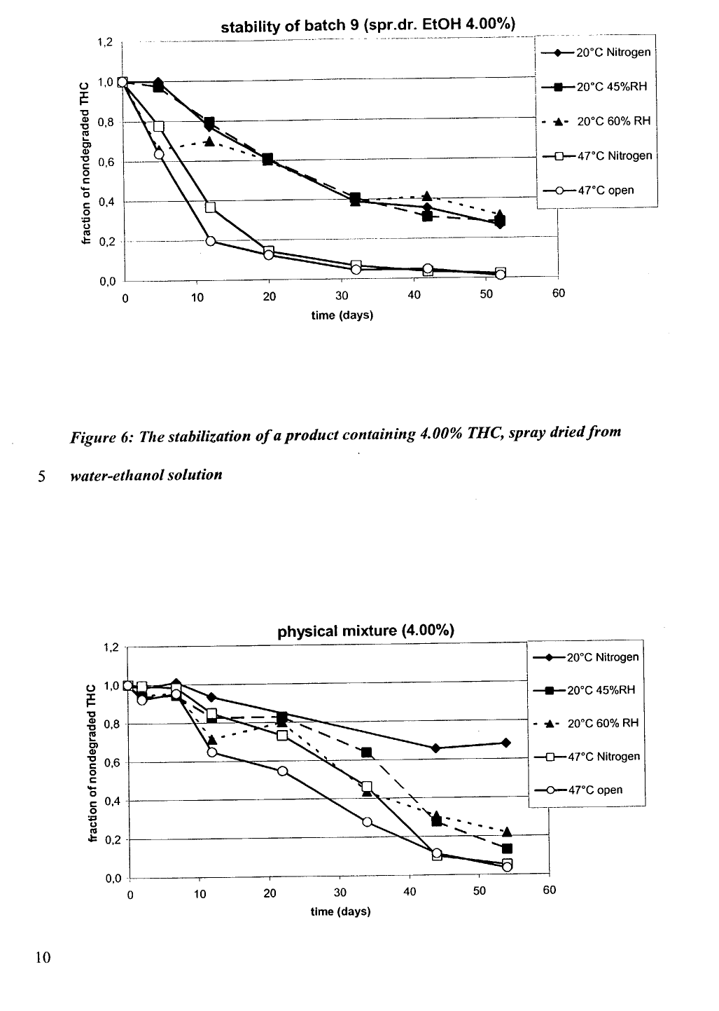

The freeze dried sample (batch 12) is depicted in FIG. 3. Next to the five climates described before, some samples of this batch were exposed to 60° C. 0% RH. FIG. 4 shows the stability data of the batch that was spray dried from a solution of 1-PrOH and water, containing 3.34% THC, FIG. 5 shows a batch with a higher THC content; 7.77% but also spray dried from a water-1-propanol solution. FIG. 6, shows the stability data of the batch that was spray dried from a solution of ethanol and water, containing 4.00% THC.

The results from the spray dried batches show that the stability of the THC is improved by the formulation. The temperature has the biggest influence on the degradation rate. Moisture and oxygen are of less importance. However, it should be noticed that the samples stored under nitrogen were probably contaminated with oxygen to a certain level.

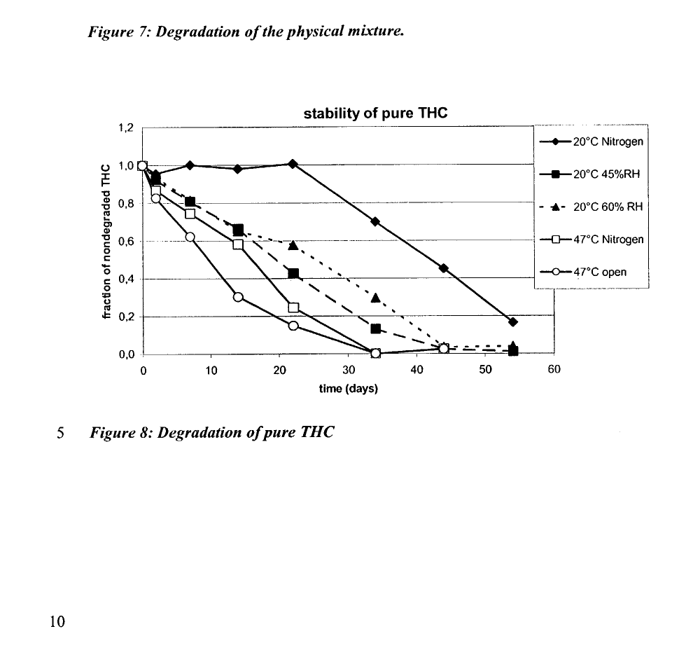

The different figures clearly show that the stability of the freeze dried product was superior, when compared with the physical mixture and the pure THC (see FIGS. 7 and 8). Apparently, the process by which the sugar glasses are prepared strongly influences the stability of the product.

As can be seen in FIG. 5 the degradation in the freeze dried product is minimal for all tested conditions, except for 60% RH. However the somewhat lower concentration found here might also be caused by the fact that at this condition the material is collapsed which makes the extraction procedure less effective.

Reference Batches

To test the stabilizing capacity of inulin, the data shown above should be compared with a batch with the same chemical and physical structure, but without the inulin. This would imply that a reference batch consists of separate inulin molecules, in fact a vapour of THC. Because this is impractical, two other reference-batches are prepared: a physical mixture containing about 4% THC and 96% unprocessed inulin and pure THC. The results are presented in FIGS. 7 and 8 respectively.

It has to be mentioned that during the preparation of the physical mixture the solution of THC in methanol softened the inulin powder to some extend. After evaporation of the methanol, a more or less solid film of inulin and THC appeared at the bottom of the vial. The low porosity film causes an extra protection of this reference material. Besides that, it is possible that the mixing of the methanolic THC solution with the sugar already results in inclusion of a part of the THC.

It should be emphasized that the self protection is also relevant in the pure THC samples since they form also a shielding film.

The contents of all cited references throughout this application are hereby expressly incorporated by reference. The practice of the present invention will employ, unless otherwise indicated, conventional techniques of pharmacology and pharmaceutics, which are within the skill of the art.

Although the invention has been described with respect to specific embodiments and examples, it should be appreciated that other embodiments utilizing the concept of the present invention are possible without departing from the scope of the invention. The present invention is defined by the claimed elements, and any and all modifications, variations, or equivalents that fall within the true spirit and scope of the underlying principles.

What is claimed is:

1. A pharmaceutical composition comprising a natural cannabinoid compound and at least one of a glass of a sugar, a sugar alcohol, a mixture of sugars or a mixture of sugar alcohols, wherein the natural cannabinoid compound is incorporated in the sugar glass as a monomolecular encapsulation without formation of a guest-host complex.

2. The pharmaceutical composition of claim 1, wherein the sugar or mixture of sugars comprises a non-reducing sugar or a mixture of non-reducing sugars.

3. The pharmaceutical composition of claim 1, wherein the natural cannabinoid compound comprises Δ⁹-tetrahydrocannabinol, or a salt, an ester, an amide, an enantiomer, an isomer, a tautomer, a prodrug, or a derivative thereof.

4. The pharmaceutical composition of claim 1, wherein the sugar glass comprises a glass transition temperature of above about 50° C. at normal environmental conditions.

5. The pharmaceutical composition of claim 1, wherein the sugar or mixture of sugars comprises a fructane or a mixture of fructanes.

6. The pharmaceutical composition of claim 5, wherein the fructane or mixture of fructanes comprises at least one of: (a) inulin; (b) a mixture of inulins; (c) inulin with a DP of greater than about 6; or (d) a mixture of inulins wherein each inulin has a DP of greater than about 6.

7. The pharmaceutical composition of claim 6, wherein the inulin or each inulin in the mixture comprises a DP of between about 10 and about 30.

8. The pharmaceutical composition of claim 1 in the form of a tablet comprising a normal oral tablet, a sublingual tablet, a buccal tablet or an orally disintegrating or dissolving tablet, a capsule, a lozenge, an enema, a suppository, a product for transdermal administration, a powder for pulmonary administration, a suspension for pulmonary administration, or a rod or suspension for subcutaneous or intramuscular administration.

9. The pharmaceutical composition of claim 8 intended for oral administration.

10. The pharmaceutical composition of claim 8 intended for pulmonary administration.

11. A method of preparing a pharmaceutical composition comprising a natural cannabinoid compound and a glass of a sugar, a sugar alcohol, a mixture of sugars or a mixture of sugars alcohols, wherein the natural cannabinoid compound is incorporated in the sugar glass as a monomolecular encapsulation without formation of a guest-host complex; the method comprises
a) dissolving the natural cannabinoid compound in an organic solvent that is soluble in water and dissolving the sugar, sugar alcohol, mixture of sugars or mixture of sugar alcohols in water;
b) mixing the dissolved cannabonoid compound and the dissolved sugar, sugar alcohol, mixture of sugars or mixture of sugar alcohols to obtain a sufficiently stable mixture; and
c) drying the mixture by freeze drying, spray drying, vacuum drying, or super critical drying.

12. The method of claim 11, wherein the sugar or mixture of sugars comprises a non-reducing sugar or a mixture of non-reducing sugars.

13. The method of claim 11, wherein the natural cannabinoid compound comprises Δ⁹-tetrahydrocannabinol.

14. The method of claim 11, wherein the sugar or mixture of sugars comprises a fructane or a mixture of fructanes.

15. The method of claim 14, wherein the fructane or mixture of fructanes comprises at least one of: (a) inulin; (b) a mixture of inulins; (c) inulin with a DP of greater than about 6; or (d) a mixture of inulins wherein each inulin has a DP of greater than about 6.

16. The method of claim 11, wherein the organic solvent comprises a C₁-C₆ alcohol.

17. The method of claim 11, wherein the organic solvent comprises a C₂-C₄ alcohol.

18. The method of claim 11, wherein the alcohol comprises ethanol, or n-propanol.

19. The method of claim 11, wherein the alcohol comprises t-butyl alcohol.

20. The method of claim 11, wherein the pharmaceutical composition is prepared by freeze drying.

21. The method of claim 11, wherein the pharmaceutical composition is further processed into a tablet comprising a normal oral tablet, a sublingual tablet, a buccal tablet or an orally disintegrating or dissolving tablet, a capsule, a lozenge, an enema, a suppository, a product for transdermal administration, a powder for pulmonary administration, a suspension for pulmonary administration, or a rod or suspension for subcutaneous or intramuscular administration.

22. A method for preventing or treating a disorder that is responsive to a cannabinoid in a subject in need thereof, comprising: administering to the subject a pharmaceutical composition of any of claims 1-10.

23. A method for preventing or treating a disorder where a cannabinoid is indicated, the method comprises administering the composition according to any of claims 1-10 to a subject in need of such prevention or treatment.
